(19) United States
(12) Patent Application Publication
(10) Pub. No.: US 2026/0256354 A1
(43) Pub. Date: Sep. 3, 2026

(54) DEVICE FOR MEASURING THE EYE LENGTH

(71) Applicant: Heidelberg Engineering GmbH, Heidelberg (DE)

(72) Inventor: Christoph BROSCHE, Eppelheim (DE)

(73) Assignee: Heidelberg Engineering GmbH, Heidelberg (DE)

(21) Appl. No.: 19/141,092

(22) PCT Filed: Sep. 21, 2023
(86) PCT No.: PCT/EP2023/076071

§ 371 Date: Jun. 19, 2025

(30) Foreign Application Priority Data
Dec. 19, 2022 (DE) 10 2022 133 951.3

(51) Int. Cl. A61B 3/10; A61B 3/14; G01B 9/02015; G01B 9/02091

(52) U.S. Cl. CPC A61B 3/102; A61B 3/14; G01B 9/02028; G01B 9/02091; A61B 3/1005

(57) ABSTRACT

With regard to the problem of specifying a device that permits a simple and robust measurement of the eye length, in particular without hardware modification on a camera, an optical device for use in an apparatus (13) for carrying out optical coherence tomography (OCT), comprising a cylindrical optical channel (1) for guiding and refracting light, which has a first diameter (1a) and a first length (1b), is characterised in that the first channel (1) is, in some sections, surrounded annularly and concentrically by a second optical channel (2) for guiding and refracting light, the second channel (2) having a larger diameter (2a) but a shorter length (2b) than the first channel (1).

The invention relates to an optical device according to the preamble of claim 1.

The term optical coherence tomography (usually abbreviated to OCT) refers to an imaging method. With this method, two-dimensional and three-dimensional images of light-scattering structures may be obtained. In this method, light having a certain bandwidth is usually split into two partial beams in a beam splitter. The first partial beam strikes the specimen to be studied, or the object, and the second partial beam travels along a reference path. The light reflected by the specimen, or the object, interferes with the reference beam. By using signals from the interference, the specimen may be studied with depth resolution, i.e. in the depth of the optical axis of the first partial beam, by so-called A-scans. In addition, it is also possible to scan the specimen in a plane, or laterally, with the first partial beam in order to obtain OCT images. B-scans are composed of a plurality of A-scans.

Methods for measuring or estimating eye lengths by means of the aforementioned OCT technology are already known.

In some methods of this type, a relatively broad OCT spectrum is necessary. In other methods, additional optical components are necessary. Many methods are time-consuming or affected by errors due to eye movements.

The object of the invention is therefore to provide a device that allows simple and robust measurement of eye lengths, in particular without hardware modifications to a camera.

The present invention achieves the aforementioned object by the features of claim 1.

According to the invention, it has been discovered that providing two optical channels having different diameters and lengths makes it possible that, besides two focal planes, it is also possible to generate two OCT reference planes having the same optical path length. By using these planes, an eye length measurement may optionally take place without using a beam splitter, in particular a conventional beam splitter, in the optical device or in an apparatus in which the device is installed. The described device does not require a broad OCT spectrum, and may be used in many already existing OCT apparatuses. Also, hardware modifications to cameras of existing OCT apparatuses are not necessary in order to carry out, in particular, an eye length measurement with the device. Moreover, both the optical device and the eye length measurement carried out therewith are very robust and precise. The retina and the cornea may be imaged simultaneously and sharply during use of the optical device or the eye length measurement carried out therewith.

The two optical channels could be formed by lenses, which are arranged in succession and collinearly and concentrically in relation to their respective optical axis, at least one lens having a larger diameter than another lens and thereby contributing to the second channel with its material part that protrudes annularly beyond the other lens, and thus forming an annular channel. Lenses are robust and may readily be connected mechanically or materially to one another, or optically connected in series. Moreover, the optical properties of lenses are clearly defined by their refractive behavior and are substantially free from system influences, for instance heating or cooling. In such an optical device, central light rays travel through all the lenses, while outer light rays travel only through a lens having a large diameter.

A plurality of lenses having the same second diameter could lie as a cluster between at least two lenses having the same first diameter, the first diameter being less than the second diameter. By the use of a plurality of lenses as a cluster, the refractive behavior of the cluster as a whole may be adjusted.

The optical device could have two focal planes, namely a first focal plane for light that travels through the first, inner channel and a second focal plane for light that travels only through the annular second channel. Central or outer light rays are in each case focused in the respective focal plane. The optical device may be designed so that a focal plane for the central light rays lies on the cornea and a focal plane for the outer light rays lies on the retina.

The optical device could have two reference planes, preferably having the same overall optical path length, namely a first reference plane for light that travels through the first, inner channel, which forms a longer optical path for light, and a second reference plane for light that travels only through the annular second channel, which forms a shorter optical path. Ultimately, the optical path length as far as the two reference planes is identical, and only a partial path, namely a first length, is significantly longer for the central beam as a function of the refractive index of the glass. The optical path length for the central light rays is significantly longer, which gives rise to different reference planes. The optical device may be designed so that the reference plane of the central light rays lies on the cornea and the reference plane of the outer light rays lies on the retina.

The splitting into two focal or reference regions preferentially takes place via concentric optics having different diameters. The central light rays of a scan field travel through all the optics, while marginal light rays do not travel through all the optics.

The optical device may be configured as an objective or replaceable objective. An objective forms a prefabricated structural unit. By replaceability of the objective, an OCT apparatus may be retrofitted. Such an objective may be used for eye length measurement. In particular, such an objective may be configured as a multifocal objective.

In a method for determining separations of two structures, in which an optical device of the type described here is used, the first structure could be recorded in a first focal plane and/or first reference plane of the optical device, the second structure being recorded in a second focal plane and/or second reference plane of the optical device, in which case the separation of the structures is determined from data of its recording and/or from an image representation of the two structures.

The first structure could be the cornea or a region of the cornea of an eye, and the second structure could be the retina or a region of the retina of the eye. This provides a method for measuring eye lengths by means of OCT technology.

An apparatus for carrying out an optical coherence tomography (OCT), which comprises an optical device of the type described here, could have an electronic device, in particular as hardware with corresponding software, which performs the method described here for determining separations of two structures.

The electronic device controls the optics, in particular an interferometer, of the apparatus in order to record the separations of the structures by means of the optical device. Further, the electronic device determines the separations of the recorded structures by algorithms automatically or in a manner defined by the user. The electronic device could represent the recorded structures in an image. The user may ascertain information that is helpful for a diagnosis with the aid of the image. The images may be stored in a memory of the electronic device and/or processed.

In the drawing,

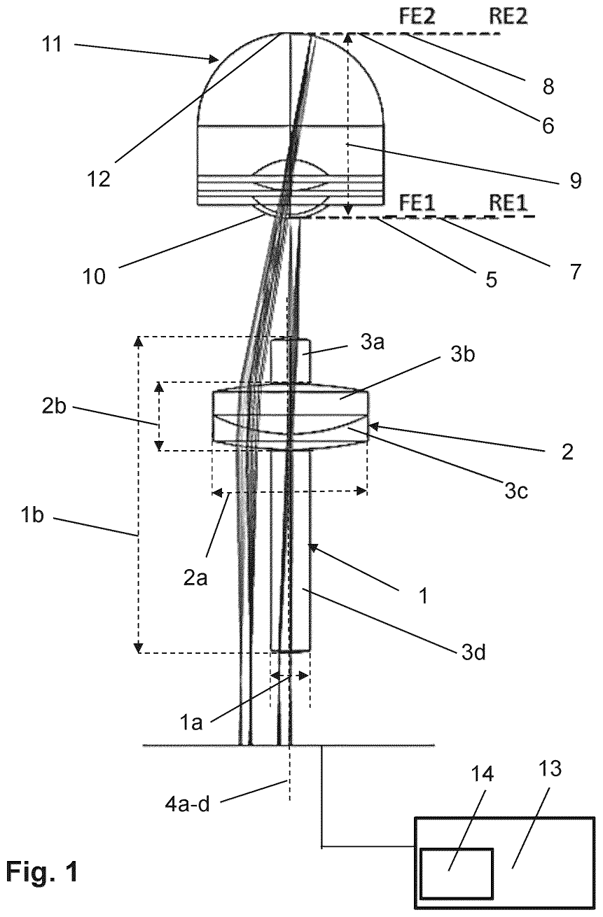

FIG. 1 shows a schematic representation of an optical arrangement, in which an optical device for use of the eye length measurement by means of OCT technology is employed, and

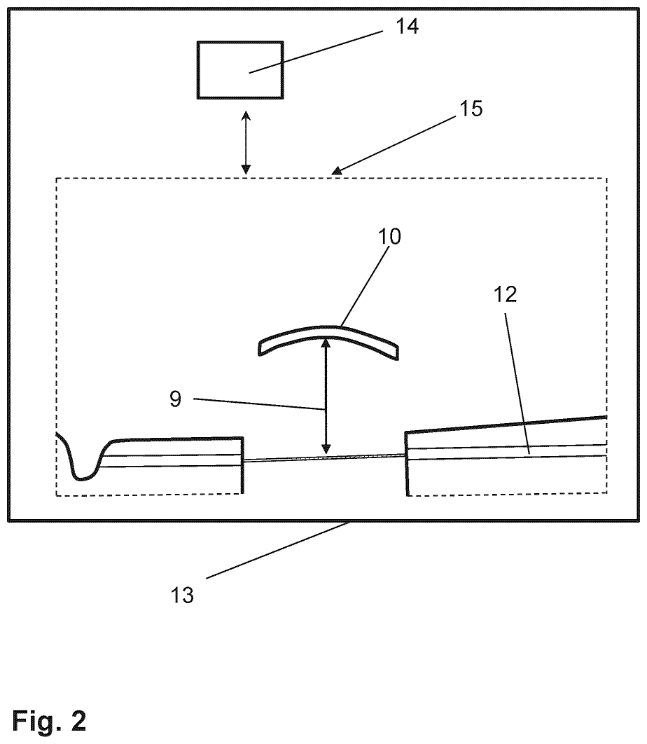

FIG. 2 shows a sectional image, which represents that the cornea is imaged sharply in a central region and the retina is imaged sharply in a marginal region.

FIG. 1 shows an optical device for use in an apparatus 13 for carrying out an optical coherence tomography. The optical device comprises or forms a cylindrical optical channel 1 for guiding and refracting light, which has a first diameter 1a and a first length 1b. The first channel 1 is annularly and concentrically enclosed locally by a second optical channel 2 for guiding and refracting light, the second channel 2 having a larger diameter 2a but a shorter length 2b than the first channel 1.

The two channels 1, 2 are formed by lenses 3a-d, which are arranged in succession and collinearly and concentrically in relation to their respective optical axis 4a-d. Specifically, a single optical axis is therefore provided, with which the optical axes 4a-d coincide. At least one of the lenses 3b, 3 c has a larger diameter than another lens 3a, 3d and thereby contributes to the second channel 2 with its material part that protrudes annularly beyond the other lens, and thus forms an annular channel.

Specifically, two lenses 3b, 3c having the same second diameter 2a lie as a cluster between two lenses 3a, 3d having the same first diameter 1a, the first diameter 1a being less than the second diameter 2a.

The optical device has two focal planes 5, 6, namely a first focal plane 5 for light that travels through the first, inner channel 1 and a second focal plane 6 for light that travels only through the annular second channel 2.

The optical device has two reference planes 7, 8, namely a first reference plane 7 for light that travels through the first, inner channel 1, which forms a longer optical path for light, and a second reference plane 8 for light that travels only through the annular second channel 2, which forms a shorter optical path. The device is configured as an objective or replaceable objective.

FIG. 1 also shows with the aid of the arrangement a method for determining separations 9 of two structures, in which an optical device of the type described above is used, the first structure being recorded in a first focal plane 5 and first reference plane 7 of the optical device, the second structure being recorded in a second focal plane 6 and second reference plane 8 of the optical device, and the separation 9 of the structures being determined from data of its recording and/or from an image representation of the two structures.

In the specific but not restrictive exemplary embodiment, the first structure is the cornea 10 or a region of the cornea 10 of an eye 11, and the second structure is the retina 12 or a region of the retina 12 of the eye 11.

FIG. 1 in this regard specifically shows the optical structure of an objective, specifically of an OCT objective for measuring the eye length of a human eye 11. The objective consists of a plurality of lenses 3a-d, which are arranged concentrically in series and have different diameters. Thus, the central light rays as represented in FIG. 1 travel through all the optics, while the outer light rays travel only through optics having a large diameter. This gives rise to different focal planes 5, 6 for central and outer light rays. Also, the optical path length for the central light rays is significantly longer, which also gives rise to different reference planes 7, 8 having the same overall optical path length. The design of the objective is such that the focal plane 6 and the reference plane 8 of the outer rays lie on the retina 12. The focal plane 5 and the reference plane 7 of the central rays lie on the cornea 10.

FIG. 1 schematically shows an apparatus 13 for carrying out an optical coherence tomography (OCT), comprising the optical device and an electronic device 14, which performs the method described above.

In an OCT method, light is usually split into two partial beams. A first partial beam strikes the specimen to be studied, for example the cornea 10 or the retina 12, and the second partial beam travels along a reference path. The respective first partial beam, specifically, is guided onto the respective focal plane 5, 6. The respective second partial beam travels along a reference path of finite length. The position of the reference plane, in which the OCT image is represented, is defined by the reference branch length. The “optical path lengths” of RE1 (reference plane 7) and RE2 (reference plane 8) are equally long, so that the two regions can be represented simultaneously in the OCT image. Specifically, the respective reference plane 7, 8 coincides with the respective focal plane 5, 6. A light beam reflected by the specimen interferes with the reference beam. By using signals from the interference, the specimen may be studied with depth resolution, i.e. in the depth of the optical axis of the first partial beam, by so-called A-scans.

FIG. 2 shows the result of an application of the method described with reference to FIG. 1 to a human eye, the first structure being the cornea 10 or a region of the cornea 10 of an eye 11 and the second structure being the retina 12 or a region of the retina 12 of the eye 11. FIG. 2 shows that the electronic device 14 represents the structures in an image 15. FIG. 2 shows the image 15 as a sectional image. If a straight line scan (OCT B-scan) is carried out radially through the optical axis 4a-d of the objective, or of the optical device, this gives rise to the sectional image according to FIG. 2. The cornea 10 is imaged sharply in the central region, and the retina 12 is imaged sharply in the marginal region. By means of the separation 9 of the structures in the sectional image, the distance between the cornea 10 and the retina 12 may be calculated accurately.

Determination of the exact eye length offers certain advantages. If the eye length is known, for example, scalings may be calculated more accurately and structures of an eye may be represented to scale.

### LIST OF REFERENCE SIGNS

1 cylindrical optical channel
1a first diameter
1b first length
2 second optical channel
2a shorter length
2b larger diameter
3a-d lenses
4a-d optical axis
5 first focal plane
6 second focal plane
7 first reference plane
8 second reference plane
9 separation
10 cornea
11 eye
12 retina
13 OCT apparatus
14 electronic device
15 image

1. An optical device for use in an apparatus (13) for carrying out an optical coherence tomography (OCT), comprising a cylindrical optical channel (1) for guiding and refracting light, which has a first diameter (1a) and a first length (1b), characterized in that the first channel (1) is annularly and concentrically enclosed locally by a second optical channel (2) for guiding and refracting light, the second channel (2) having a larger diameter (2a) but a shorter length (2b) than the first channel (1).

2. The device as claimed in claim 1, characterized in that the two channels (1, 2) are formed by lenses (3a-d), which are arranged in succession and collinearly and concentrically in relation to their respective optical axis (4a-d), at least one lens (3b, 3c) having a larger diameter than another lens (3a, 3d) and thereby contributing to the second channel (2) with its material part that protrudes annularly beyond the other lens, and thus forming an annular channel.

3. The device as claimed in claim 2, characterized in that a plurality of lenses (3b, 3c) having the same second diameter (2a) lie as a cluster between at least two lenses (3a, 3d) having the same first diameter (1a), the first diameter (1a) being less than the second diameter (2a).

4. The device as claimed in claim 3, characterized by two focal planes (5, 6), namely a first focal plane (5) for light that travels through the first, inner channel (1) and a second focal plane (6) for light that travels only through the annular second channel (2).

5. The device as claimed in claim 3, characterized by two reference planes (7, 8), namely a first reference plane (7) for light that travels through the first, inner channel (1), which forms a longer optical path for light, and a second reference plane (8) for light that travels only through the annular second channel (2), which forms a shorter optical path.

6. The device as claimed in claim 3, characterized by a configuration as an objective or replaceable objective.

7. A method for determining separations (9) of two structures, in which an optical device as claimed in claim 3 preceding claims is used, wherein the first structure is recorded in a first focal plane (5) and/or first reference plane (7) of the optical device, wherein the second structure is recorded in a second focal plane (6) and/or second reference plane (8) of the optical device, and wherein the separation (9) of the structures is determined from data of its recording and/or from an image representation of the two structures.

8. The method as claimed in claim 7, characterized in that the first structure is the cornea (10) or a region of the cornea (10) of an eye (11), and in that the second structure is the retina (12) or a region of the retina (12) of the eye (11).

9. An apparatus (13) for carrying out an optical coherence tomography (OCT), comprising an optical device as claimed in claim 3 and an electronic device (14) which performs a method for determining separations (9) of two structures, wherein the first structure is recorded in a first focal plane (5) and/or first reference plane (7) of the optical device, wherein the second structure is recorded in a second focal plane (6) and/or second reference plane (8) of the optical device, and wherein the separation (9) of the structures is determined from data of its recording and/or from an image representation of the two structures.

10. The apparatus as claimed in claim 9, characterized by an electronic device (14) that represents the structures in an image (15).

11. The apparatus of claim 9, wherein the first structure is the cornea (10) or a region of the cornea (10) of an eye (11), and in that the second structure is the retina (12) or a region of the retina (12) of the eye (11).
